(19) United States
(12) Patent Application Publication
(10) Pub. No.: US 2021/0236861 A1
(43) Pub. Date: Aug. 5, 2021

(54) PORTABLE ULTRASONIC STIMULATOR

(71) Applicant: Su Gun LEE, Paju-si (KR)

(72) Inventor: Joo Hyun KIM, Seoul (KR)

(73) Assignee: Su Gun LEE, Paju-si (KR)

(21) Appl. No.: 17/224,820

(22) Filed: Apr. 7, 2021

Related U.S. Application Data
(63) Continuation of application No. PCT/KR2019/012823, filed on Oct. 1, 2019.

(30) Foreign Application Priority Data
Oct. 8, 2018 (KR) 10-2018-0119543

(51) Int. Cl. A61N 7/00

(52) U.S. Cl. CPC A61N 7/00; A61N 2007/0073; A61N 2007/0034

(57) ABSTRACT

The present invention relates to a portable ultrasonic stimulator, and the purpose of the present invention is to provide a portable ultrasonic stimulator which alternately outputs low-frequency ultrasound having the high effect of increasing temperature in the skin and high-frequency ultrasound inducing generation of anti-aging substances in the skin, thereby providing a high skin-tightening effect.

### CROSS-REFERENCE TO RELATED APPLICATIONS

The present application is a continuation of International Patent Application No. PCT/KR2019/012823, filed on Oct. 1, 2019, which is based upon and claims the benefit of priority to Korean Patent Application No. 10-2018-0119543 filed on Oct. 8, 2018. The disclosures of the above-listed applications are hereby incorporated by reference herein in their entirety.

### BACKGROUND

Embodiments of the inventive concept described herein relate to a portable ultrasonic stimulator, and more particularly, relate to an apparatus for adjusting a frequency of an ultrasonic wave and stimulating the skin.

In general, because a skin care state is an important element in appearance, women have enormous interest in skin care. Recently, there has been an increase in skin care among men.

Meanwhile, various cosmetics are used as a means for skin care, and a skin massage is also becoming a means of skin care. In this case, because it is able for you to do the skin care massage by yourself, but the skin massage that you may do yourself is only basic, recently, many people visit specialized hospitals or specialized shops that use various types of skin massagers and skin treatment devices to receive skin care massages.

As such, when you visit the skin specialty hospital or the skin massage shop to receive a massage, you may receive several types of skin massages by means of various skin massagers and skin treatment devices, so it is more satisfying than doing the skin massage yourself.

However, when you visit the skin specialty hospital or the skin massage shop to receive a massage, it takes a considerable amount of time to travel and there is a burden on the cost.

To address the above problems, recently, various types of skin massagers capable of being conveniently used by the user have been proposed. Because the skin massager that the user may use conveniently is not only effective compared to the manual skin massage, but may also reduce the time and cost burden associated with the visit to the skin specialty hospital or skin massage shop, it is rapidly spreading.

Meanwhile, an ultrasonic wave is a sound that vibrates so fast that humans cannot hear it, and technically refers to a sound that vibrates more than 20,000 times per second. Because the ultrasonic wave has essentially the same properties as sound waves in the audible range, but has a short wavelength, it generates a fairly strong vibration and has a strong force to shake a material. Such an ultrasonic wave has the properties of radio waves and light other than the properties of sound. The ultrasonic wave is used in various fields such as ultrasonic cleaners, ultrasonic humidifiers, fish finders, ultrasonic welding, ultrasonic tomography diagnostics, and ultrasonic diagnostic devices. Particularly, when the ultrasonic wave is conducted to the human body, because it reaches a depth of several centimeters (4 to 5 cm) and its effect shows the same therapeutic effect as massage, the ultrasonic wave is applied to an ultrasonic therapy device used for treatment of arthritis, rheumatism, back pain, post-traumatic pain, neuralgia, or the like as one of the medical physiotherapy treatments.

When the skin is irradiated with a soft ultrasonic wave that is optimal for the skin (the vibration may not be felt by the skin, but may have a very rapid intensity of 1 million Hz), the ultrasonic wave acts as a massage of applying instantaneous pressure fluctuations to blood vessels, cells, and lymphatic vessels on the skin surface or up to 4 to 5 cm under skin to massage at high speed, the principle of the ultrasonic skin massager is to activate the skin by the promotion of skin tissue metabolism using a warming process of warming the deep areas of the skin to promote the flow of blood or lymph at the same time as a cleansing process of removing impurities in the deep pores. In general, as a ceramic ultrasonic vibration element (vibration probe) is directly attached to the probe head made of a metal case, the ultrasonic skin massager applies an ultrasonic wave of a certain frequency during a set certain time to output oscillation, and applies the converted vibration to treatment or care of the whole body of the human body or the skin of the face.

However, because an existing skin massager or an existing skin treatment device that uses one ceramic ultrasonic vibration element emits only an ultrasonic wave of one frequency band among ultrasonic waves with several types of frequency bands, several types of skin massagers and skin treatment devices should be provided to obtain various skin massage effects.

Furthermore, an ultrasonic wave may vary in a depth where it penetrates into skin according to a frequency and may have a difference in an influence on skin depending on a frequency. Thus, there is a need to use ultrasonic waves of several frequencies to enhance a skin tightening effect using ultrasonic waves.

### SUMMARY

Embodiments of the inventive concept provide a portable ultrasonic stimulator for providing a high skin-tightening effect by alternatively outputting a low-frequency ultrasonic wave having a high temperature rise effect in skin and a high-frequency ultrasonic wave guiding to produce a skin aging prevention material in skin.

The technical problems to be solved by the inventive concept are not limited to the aforementioned problems, and any other technical problems not mentioned herein will be clearly understood from the following description by those skilled in the art to which the inventive concept pertains.

According to an embodiment, a portable ultrasonic stimulator may include a housing having a body part held by a user and an ultrasonic transducer combining part, an ultrasonic transducer that is provided in the ultrasonic transducer combining part and outputs an ultrasonic wave to skin by means of vibration, a controller that sets an ultrasonic wave output as providing the ultrasonic transducer with a current of a specific frequency to a specific frequency, a battery provided in the body part, and a charging terminal provided at one side of the housing. The controller may adjust a frequency of current during a specific entire output time to alternately provide a low-frequency ultrasonic wave and a high-frequency ultrasonic wave. The entire output time may include a first output time for providing the high-frequency ultrasonic wave and a second output time for providing the low-frequency ultrasonic wave.

Furthermore, the low-frequency ultrasonic wave may be less than 1 MHz, and the high-frequency ultrasonic wave may be greater than or equal to 1 MHz.

Furthermore, the high-frequency ultrasonic wave may be provided to accelerate to produce a skin aging prevention material in skin and decelerate to produce a skin aging acceleration material in skin, and the low-frequency ultrasonic wave may be provided to provide a temperature rise effect of a skin layer.

Furthermore, the controller may provide the ultrasonic transducer with current intensity in which the high-frequency ultrasonic wave penetrates into the dermal layer.

Furthermore, the entire output time may be set such that the sum of the second output time is longer than the sum of the first output time. The ultrasonic transducer may have maximum efficiency in the low-frequency ultrasonic wave.

Furthermore, the ultrasonic transducer may be replaced and combined to the ultrasonic transducer combining part, such that a type of an ultrasonic wave output to skin is set.

Furthermore, the portable ultrasonic stimulator may further include an ultrasonic generator. The ultrasonic transducer may be selected among a point-focused ultrasonic transducer, a line-focused ultrasonic transducer, and a planar ultrasonic transducer.

Furthermore, the planar ultrasonic transducer may be included in the ultrasonic generator. A surface of the ultrasonic generator may be formed in a form being in direct contact with the skin of the user. The planar ultrasonic transducer may vibrate to output an ultrasonic wave of an unfocused form to the outside of the ultrasonic generator to be provided into the skin being in contact with the ultrasonic generator.

Furthermore, the point-focused ultrasonic transducer may be formed in a shape, a cross section of which has a specific curvature, in the ultrasonic generator. The ultrasonic generator may allow an ultrasonic wave generated by the point-focused ultrasonic transducer to be focused on a center point according to the curvature of the point-focused ultrasonic transducer.

Furthermore, the line-focused ultrasonic transducer may be formed in a shape, a cross section of which has a specific curvature, in the ultrasonic generator. The ultrasonic generator may allow an ultrasonic wave generated by the line-focused ultrasonic transducer to be focused on a specific point on a line, the specific point being a center point of the cross section.

Furthermore, the point-focused ultrasonic transducer or the line-focused ultrasonic transducer may include a cartridge housing, an acoustic liquid, and an acoustic window. The acoustic liquid may function as a medium allowing an ultrasonic wave generated by the point-focused ultrasonic transducer or the line-focused ultrasonic transducer to be delivered into a skin tissue of the user. The acoustic window may allow the acoustic liquid to be sealed in the cartridge housing and may play a role as a path where an ultrasonic wave provided by the point-focused ultrasonic transducer or the line-focused ultrasonic transducer to the skin of the user.

Furthermore, the controller may set the entire output time when it is able to reach a tightening effect, with regard to a temperature rise by a low-frequency ultrasonic wave during one or more second output times included in the entire output time and a temperature rise by a high-frequency ultrasonic wave during one or more first output times included in the entire output time.

Furthermore, the controller may set the entire output time based on a high-frequency ultrasonic wave output condition and a low-frequency ultrasonic wave output condition to calculate the shortest time when it is able to generate a skin tightening effect at a specific location, when the user uses a manner which repeats a process of placing the ultrasonic stimulator on each location of skin during a specific time to output an ultrasonic wave and moving the ultrasonic stimulator to a next location to output the ultrasonic wave.

Furthermore, the portable ultrasonic stimulator may further include a motion sensor that measures motion of the portable ultrasonic stimulator. The controller may set the ratio of the first output time to the second output time with regard to a skin characteristic of the user and a purpose of use, when the user rubs a skin region to perform therapy or care while outputting an ultrasonic wave, may set a time length where it is able to increase the entire area of the skin region to a specific temperature or more to the entire output time, and may set the entire output time when it is able to reach a tightening effect, with regard to a temperature rise by the low-frequency ultrasonic wave during the second output time and a temperature rise by the high-frequency ultrasonic wave during the one or more first output times.

Furthermore, the controller may determine whether the entire skin region of the user is evenly rubbed based on a sensing result of the motion sensor.

In addition, another method for implementing the inventive concept, another system therefor, and a computer-readable storage medium storing a computer program for executing the method may be further provided.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a drawing illustrating the appearance of a portable ultrasonic stimulator according to an embodiment of the inventive concept;

FIG. 2 is a block diagram illustrating an internal configuration of a portable ultrasonic stimulator according to an embodiment of the inventive concept; and

FIG. 3 is a drawing illustrating the internal depth of the skin where an energy of 50% arrives according to a frequency of an ultrasonic wave.

### DETAILED DESCRIPTION

Hereinafter, an embodiment of the inventive concept will be described in detail with reference to the accompanying drawings. Advantages, features, and methods of accomplishing the same will become apparent with reference to embodiments described in detail below together with the accompanying drawings. However, the inventive concept is not limited by embodiments disclosed hereinafter, and may be implemented in various forms. Rather, these embodiments are provided to so that this disclosure will be through and complete and will fully convey the concept of the invention to those skilled in the art, and the inventive concept will only be defined by the appended claims. The same reference denotations refer to the same components throughout the specification.

Unless otherwise defined herein, all terms (including technical and scientific terms) used in the specification may have the same meaning that is generally understood by a person skilled in the art. Also, terms which are defined in a dictionary and commonly used should be interpreted as not in an idealized or overly formal detect unless expressly so defined.

Terms used in the specification are used to describe embodiments of the present disclosure and are not intended to limit the scope of the present disclosure. In the specification, the terms of a singular form may include plural forms unless otherwise specified. The expressions “comprise” and/or “comprising” used herein indicate existence of one or more other elements other than stated elements but do not exclude presence of additional elements.

Hereinafter, a detailed description of a portable ultrasonic stimulator according to embodiments of the inventive concept will be given with reference to the accompanying drawings.

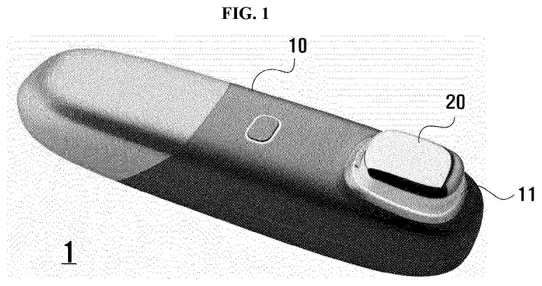

FIG. 1 is a drawing illustrating the appearance of a portable ultrasonic stimulator 1 according to an embodiment of the inventive concept.

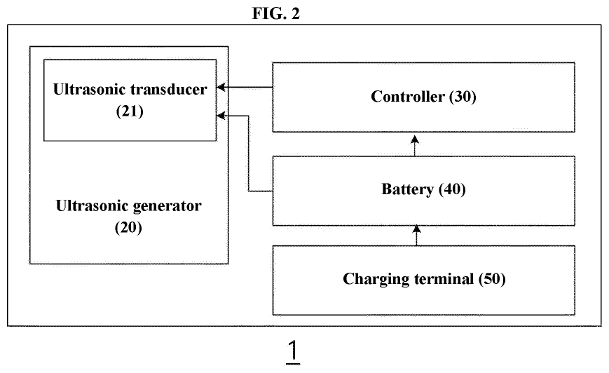

FIG. 2 is a block diagram illustrating an internal configuration of a portable ultrasonic stimulator 1 according to an embodiment of the inventive concept.

Referring to FIGS. 1 and 2, the portable ultrasonic stimulator 1 may include all or some of a body housing 10, an ultrasonic transducer 21, a controller 30, a battery 40, and a charging terminal 50. Hereinafter, a detailed description of each operation will be given.

The body housing 10 may play a role in including the components therein. As an embodiment, the body housing 10 may include a body part and an ultrasonic transducer combining part 11. As shown in FIG. 1, the body part may be a portion held by a user, which may include the controller 30, the battery 40, or the like described below therein. The ultrasonic transducer combining part 11 may be a portion with which the ultrasonic transducer 21 described below is combined. In addition, the body housing 10 may be fabricated in a shape where it is easy to be held by the user and provide skin with an ultrasonic wave.

The ultrasonic transducer 21 may be provided in the ultrasonic transducer combing part 11 and may play a role in outputting an ultrasonic wave to skin by means of vibration. In other words, the ultrasonic transducer 21 may be a ceramic element which vibrates by current supplied by the controller 30 described below.

Furthermore, as another embodiment, the ultrasonic transducer 21 may be replaced and combined with the ultrasonic transducer combining part 11 to set an ultrasonic type output to skin. For example, the ultrasonic transducer 21 may include a point-focused ultrasonic transducer, a line-focused ultrasonic transducer, a planar ultrasonic transducer, and the like and may provide skin with a suitable type of ultrasonic wave depending on a situation.

For example, the planar ultrasonic transducer 21 may be included in an ultrasonic generator 20 and may be in a form where the entire flat surface of the ultrasonic generator 20 is in direct contact with skin as shown in FIG. 1. In other words, the planar ultrasonic transducer 21 may vibrate to output an unfocused ultrasonic wave to the outside of the ultrasonic generator 20 to immediately provide the ultrasonic wave into the skin which is in contact with the ultrasonic generator 20.

Furthermore, for example, the point-focused ultrasonic transducer 21 or the line-focused ultrasonic transducer 21 may be provided in a form, a cross section of which has a specific curvature, in the ultrasonic generator 20. In other words, the point-focused transducer is formed to emit an ultrasonic wave toward one focus, and the line-focused transducer is formed to emit an ultrasonic wave to each point on one line. For example, because a cross-sectional structure of the point-focused transducer has a concave shape (e.g., a hemispherical shape), the generated ultrasonic wave may be focused on a concave center point according to a curvature of the transducer. Furthermore, for example, the line-focused transducer may be fabricated in a form where the cylinder is cut and may have a shape, a cross-sectional structure of which is concave. As a result, the line-focused transducer may focus an ultrasonic wave on a specific point on the line, which is a center point of the cross section.

The point-focused ultrasonic transducer 21 or the line-focused ultrasonic transducer 21 may form a focusing point in a specific depth of skin. In other words, the point-focused ultrasonic transducer 21 or the line-focused ultrasonic transducer 21 may focus an ultrasonic wave at a depth corresponding to a difference between a radius of a circle according to a sectional curvature of the transducer and a distance from a skin surface to the farthest point of the transducer. Thus, the ultrasonic generator 20 including the point-focused ultrasonic transducer 21 or the line-focused ultrasonic transducer 21 may be fabricated in the form of a cartridge capable of being replaced according to a depth at which an ultrasonic wave is focused in skin.

Furthermore, as the point-focused ultrasonic transducer 21 or the line-focused ultrasonic transducer 21 is fabricated in the form of the cartridge, it may include a cartridge housing, an acoustic liquid, and an acoustic window.

The acoustic liquid may play a role in filling an internal space of the cartridge housing. In other words, the acoustic liquid may function as a medium which transmits the ultrasonic wave generated by the ultrasonic transducer 21 into skin tissues of a procedure target. It is preferable that the acoustic liquid is degassed water.

The acoustic window may allow the acoustic liquid to be sealed in the cartridge housing and may play a role as a path where the ultrasonic wave provided by the point-focused transducer or the line-focused transducer is provided to skin. In other words, the acoustic window may be formed to have an output surface aligned with the ultrasonic transducer 21. Furthermore, the acoustic window may play a role in allowing the ultrasonic wave provided by the ultrasonic transducer 21 progresses through the acoustic liquid and immediately progresses to skin, as in perfect contact with the skin.

The ultrasonic transducer 21 in the ultrasonic generator 20 may have different impedances depending on ceramic characteristics (i.e., materials), the appearance of the transducer, a thickness of the transducer, a frequency provided to the transducer, or the like. When a type of the ultrasonic transducer 21 is changed, the ultrasonic transducer 21 combined to the body housing 10 may be driven irrespective of its own impedance by the controller 30 described above.

Furthermore, as another embodiment, the ultrasonic transducer 21 may have maximum efficiency in the low-frequency ultrasonic wave. The ultrasonic transducer 21 may vary in a frequency having maximum efficiency with ceramic characteristics. In other words, a specific transducer may output an ultrasonic wave within a wide frequency range, but may have a difference in efficiency where an ultrasonic wave of each frequency is output. As will be described below, when the controller 30 provides the ultrasonic transducer 21 with a high-frequency pulse outputting a high-frequency ultrasonic wave and a low-frequency ultrasonic wave, because the low-frequency ultrasonic wave having a good temperature rise effect in skin is provided into the skin at high power efficiency to increase the inside of skin to a desired temperature within a quick time, the ultrasonic stimulator 1 may include a transducer which has high output efficiency for the high-frequency pulse for outputting the low-frequency ultrasonic wave. As a result, the portable ultrasonic stimulator 1 may provide a desired temperature rise effect within a short time.

The controller 30 may play a role in setting an ultrasonic wave output as providing the ultrasonic transducer 21 with a current of a specific frequency to the specific frequency. In other words, the controller 30 may play a role in providing the ultrasonic transducer 21 with a specific high-frequency pulse.

The controller 30 may deliver a high-frequency pulse for providing a high-frequency ultrasonic wave or a low-frequency ultrasonic wave to the ultrasonic transducer 21. For example, the low-frequency ultrasonic wave may be less than 1 MHz, and the high-frequency ultrasonic wave may be greater than or equal to 1 MHz. Particularly, because the higher the high-frequency ultrasonic wave is in frequency, the higher the effect of controlling production of materials associated with skin aging, as will be described below, the high-frequency ultrasonic wave may be used as 10 MHz or more.

The low-frequency ultrasonic wave may play a role in increasing a temperature of skin. The inside of skin should increase to a specific temperature or more to have an effect such as skin tightening, and the low-frequency ultrasonic wave which has a better effect of increasing a temperature than the high-frequency ultrasonic wave may be used to increase a skin temperature. Furthermore, because it is able to penetrate into a deeper location in the same energy condition than the high-frequency ultrasonic wave, the low-frequency ultrasonic wave may increase a temperature of the overall dermal layer or a superficial musculo-aponeurotic system (SMAS) layer to which the effect by the ultrasonic wave should be provided.

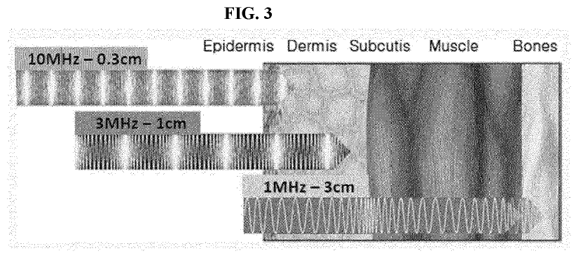

The high-frequency ultrasonic wave may play a role in adjusting production of a material associated with skin aging. The material associated with the skin aging may be a material corresponding to an extracellular matrix (ECM), which may include a skin aging acceleration material for accelerating skin aging and a skin aging prevention material for preventing skin aging. The high-frequency ultrasonic wave may play a role in decelerating to produce a skin aging acceleration material as stimulating a skin cell and accelerating to produce a skin aging prevention material. As the higher the high-frequency ultrasonic wave is in frequency, the higher the effect of decreasing the skin-aging acceleration material and increasing the skin-aging prevention material may be as the high-frequency ultrasonic wave stimulates the overall skin cell. For example, as shown in FIG. 3, the high-frequency ultrasonic wave of 10 MHz or more may be delivered in an energy of 50% to the dermal layer below a depth of 0.3 cm, thus not efficiently delivering energy to the entire depth of the dermal layer. However, as provided to the dermal layer, the high-frequency ultrasonic wave of 10 MHz or more may generate a skin aging prevention material. In detail, the extracellular matrix (ECM) may include matrix metalloproteinases (MMPs), heat shock proteins (HSPs), or the like, which is associated with skin aging.

The MMPs may correspond to a skin aging acceleration material. The MMPs may be peptide decomposition enzyme, which may play a role in breaking down all proteins except for cells. The continuous abnormal proliferation of the MMPs (particularly, continuous activation of MMP-2 which is gelatinase) destroys type IV collagen which is a principal component of the basal membrane. The damage to the basal membrane and the reduction of ground substances which fills a space between collagen and fiber such as elastic fiber results in the reduction of the thickness of dermis, thus causing reduced skin elasticity, the drooping skin, old wrinkles, or the like. The higher the high-frequency ultrasonic wave is in frequency, the more the production of MMPs may be degraded.

The HSPs may act as a catalyst for the important immune system of the human body, which may be activated in response to an external stimulus including a thermal stimulus. The HSPs may be present in an extracellular space as well as the cytosol in the cell and may have an influence on a physiological or pathological process. The HSPs may act as various roles, for example, the breakdown of unstable proteins, folding of proteins, the binding of which is not completed, refolding of incorrectly folded proteins, regulation of proteins playing a role as adjustment, inhibition of protein folding, a role in bring extracellular protein into an element in the cell, and a signaling system. Thus, the HSPs may prevent skin aging as activated. The higher the high-frequency ultrasonic wave is in frequency, the more the production of MMPs may be guided.

The controller 30 may increase an effect of combining a low-frequency ultrasonic wave with a high-frequency ultrasonic wave in one transducer to prevent skin aging. The low-frequency ultrasonic wave may be provided to increase the overall temperature from the surface of skin to the dermal layer. Because the high-frequency ultrasonic wave does not penetrate deep into skin and does not transfer energy to the overall dermal layer, it may have low efficiency of increasing the temperature of the skin layer, but may regulate a material associated with skin aging (i.e., decelerate to produce a skin aging acceleration material and accelerate to produce a skin aging prevention material). As a result, the ultrasonic stimulator 1 may increase the temperature of the skin layer using the low-frequency ultrasonic wave and the high-frequency ultrasonic wave to damage and regenerate collagen depending on degradation in tissue and may control a material associated with skin aging using the high-frequency ultrasonic wave to prevent skin aging by the ECM.

As an embodiment, the controller 30 may adjust a frequency of current during a specific entire output time to alternately provide a low-frequency ultrasonic wave and a high-frequency ultrasonic wave. In other words, the ultrasonic stimulator 1 may alternately provide the low-frequency ultrasonic wave and the high-frequency ultrasonic wave to simultaneously generate a skin tightening effect by the temperature rise and a skin aging prevention effect as the material associated with skin aging is controlled. The entire output time may include a first output time for providing the high-frequency ultrasonic wave and a second output time for providing the low-frequency ultrasonic wave. In other words, because the entire output time is a time to output the high-frequency ultrasonic wave and the low-frequency ultrasonic wave to a specific skin location to perform therapy or care, the controller 30 may divide the entire output time into the first output time and the second output time to provide the high-frequency ultrasonic wave and the low-frequency ultrasonic wave.

For example, the controller 30 may divide the entire output time into N (where N is a natural number greater than 2) and may perform one combination where the first output time and the second output time to provide the high-frequency ultrasonic wave and the low-frequency ultrasonic wave are consecutive N times. As a result, the portable ultrasonic stimulator 1 may simultaneously provide an effect of raising a temperature in skin to a specific value and an effect of producing a skin aging prevention material.

The controller 30 may adjust the length ratio of the first output time to the second output time in one combination. For example, the ratio of the first output time to the second output time may have in various manners, for example, 0.1:0.9, 0.2:0.8, 0.3:0.7, 0.4:0.6, 0.5:0.5, 0.6:0.4, 0.7:0.3, 0.8:0.2, 0.9:0.1, and the like. The ultrasonic stimulator 1 may set the ratio of the first output time to the second output time with regard to a skin characteristic of a user (e.g., a thickness of skin, skin sensitivity according to temperature rise, or the like), its purpose of use, or the like.

Furthermore, the controller 30 may adjust a time length of one combination (i.e., a combination of the first output time and a second output time) included in the entire output time depending on a skin condition of the user or a used portion to increase the number of times the high-frequency ultrasonic wave and the low-frequency ultrasonic wave are alternately provided.

For example, in the entire output time, the sum of the second output times may be set to be longer than the sum of the first output times. In other words, because the skin tightening by the temperature rise in skin is used as a main purpose to additionally obtain an effect as the skin aging prevention effect is provided, the sum of the second output times may be greater than the sum of the first output times in the entire output time. In detail, because the high-frequency ultrasonic wave provides an effect of raising temperature in skin, but is not well delivered to a deeper location than the low-frequency ultrasonic wave to have low efficiency, the controller 30 may make a time taken to provide the low-frequency ultrasonic wave long to quickly raise a skin temperature to provide the tightening effect.

Furthermore, as another embodiment, because the high-frequency ultrasonic wave also provide the effect of raising the temperature of the dermal layer, the controller 30 may set the entire output time when it is able to arrive at the tightening effect, with respect to a temperature rise by the low-frequency ultrasonic wave during one or more second output times included in the entire output time and a temperature rise by the high-frequency ultrasonic wave during one or more first output times included in the entire output time. In other words, the shorter the treatment time is on one treatment portion, the shorter the treatment time is for the entire skin. The longer the treatment time, the lower the probability that the user will spend time to use it.

For example, when using a manner where a user fixedly brings an ultrasonic therapy apparatus into contact with a specific skin location during the entire output time to move the ultrasonic therapy apparatus (i.e., a manner where the user places the ultrasonic therapy apparatus on each location of skin during a specific time to output an ultrasonic wave and move he ultrasonic therapy apparatus to a next location to output the ultrasonic wave), the controller 30 may set the entire output time with regard to a high-frequency ultrasonic wave output condition (i.e., a frequency, intensity, a first output time, or the like of a high-frequency ultrasonic wave) and a low-frequency ultrasonic wave output condition (i.e., a frequency, intensity, a second output time, or the like of a low-frequency ultrasonic wave) to calculate the shortest time when a skin tightening effect may occur at a specific location.

Furthermore, as another embodiment, when the user rubs a skin region to perform therapy or care while outputting an ultrasonic wave, the controller 30 may set a time length where it is able to increase the entire area of the skin region to a specific temperature or more to the entire output time. For example, when the user wants to treat one-sided cheek region, identifies an approximate area of the cheek region, and evenly rubs the entire cheek region, the controller 30 may calculate the shortest time when it is able to reach a specific temperature or more on the entire cheek region by means of the low-frequency ultrasonic wave and the high-frequency ultrasonic wave. Thus, the controller 30 may set a time length of the shortest time or more to the entire output time to be above a temperature where skin tightening may occur when the user evenly rubs the entire skin region.

Furthermore, as another embodiment, the ultrasonic stimulator 1 may include a motion sensor. The motion sensor may play a role in sensing whether the user evenly rubs the entire skin region. When the user rubs the cheek, the motion sensor may measure motion of the ultrasonic stimulator 1 and the controller 30 may determine whether the user evenly rubs the entire cheek. For example, the ultrasonic stimulator 1 may guide the user to start an operation of rubbing a specific point of the cheek of the user, the motion sensor may sense motion of the ultrasonic stimulator 1 according to manipulation of the user, and the controller 30 may determine whether the ultrasonic wave is evenly provided to the entire cheek as the user rubs the cheek using the ultrasonic stimulator 1.

Furthermore, as another embodiment, the controller 30 may provide the ultrasonic transducer 21 with current intensity in which the high-frequency ultrasonic wave penetrates into the dermal layer.

Furthermore, as another embodiment, the controller 30 may be composed of a circuit such that current flows in the transducer while the resistance of an input and output terminal to which the transducer is connected is close to “0”.

When an ultrasonic output part is fabricated in the form of a cartridge to be replaceable, another type of ultrasonic output part may have a different impedance. In other words, as transducers have a hemispherical form, a planar form, and a cylindrical form, respectively, each transducer may have a different impedance. Furthermore, the same type of transducer may have a different impedance by a ceramic characteristic or the like. Thus, the portable ultrasonic stimulator 1 may should be able to be run irrespective of an impedance value of the connected or combined transducer. To this end, the ultrasonic stimulator 1 may allow resistance of the input and output terminal to which the transducer is connected to be close to “0”, such that an element characteristic of the transducer available upon fabrication of the cartridge may not be limited, without the necessity of performing impedance matching when the cartridge is replaced.

The battery 40 may be provided in the body part of the body housing 10 to play a role in supplying current for driving the controller 30 and the transducer. In other words, to implement the ultrasonic stimulator 1 as a portable type, the battery 40 may be provided in the body housing 10. Furthermore, as an external power source is connected to the charging terminal 50 described below, the battery 40 may be charged.

The charging terminal 50 may be provided at one side of the body housing 10 to play a role in being connected to charge the battery 40.

According to the above embodiments of the inventive concept, the following effects may be provided.

Firstly, ultrasonic waves of various frequencies may be output using the one ultrasonic stimulator 1.

Second, the high-frequency ultrasonic wave and the low-frequency ultrasonic wave may be alternately provided to enhance the skin tightening effect. In other words, the low-frequency ultrasonic wave having high efficiency of the skin temperature rise and the high-frequency ultrasonic wave of adjusting to produce the material associated with skin aging may be alternately provided in the skin therapy process to provide both of effects by two ultrasonic waves during the entire therapy time, thus optimizing the skin tightening effect.

Finally, because it is able to replace and use the ultrasonic transducer 21 irrespective of the impedance of the ultrasonic transducer 21, there is no need to verify whether impedance corresponds to a desired range when a specific transducer is fabricated, and various types of transducers, each of which has a different impedance, may be combined and used.

According to embodiments of the inventive concept, ultrasonic waves of various frequencies may be output using the one ultrasonic stimulator.

Furthermore, according to embodiments of the inventive concept, the skin tightening effect may be enhanced as the high-frequency ultrasonic wave and the low-frequency ultrasonic wave are alternately provided. In other words, the low-frequency ultrasonic wave having high efficiency of the skin temperature rise and the high-frequency ultrasonic wave of adjusting to produce the material associated with skin aging may be alternately provided in the skin therapy process to provide both of effects by two ultrasonic waves during the entire therapy time, thus optimizing the skin tightening effect.

Furthermore, according to embodiments of the inventive concept, because it is able to replace and use the ultrasonic transducer irrespective of the impedance of the ultrasonic transducer, there is no need to verify whether impedance corresponds to a desired range when a specific transducer is fabricated, and various types of transducers, each of which has a different impedance, may be combined and used.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A portable ultrasonic stimulator, comprising:
a housing having a body part held by a user and an ultrasonic transducer combining part;
an ultrasonic transducer configured to be provided in the ultrasonic transducer combining part and output an ultrasonic wave to skin by means of vibration;
a controller configured to set an ultrasonic wave output as providing the ultrasonic transducer with a current of a specific frequency to a specific frequency;
a battery provided in the body part; and
a charging terminal provided at one side of the housing,
wherein the controller adjusts a frequency of current during a specific entire output time to alternately provide a low-frequency ultrasonic wave and a high-frequency ultrasonic wave, and
wherein the entire output time includes a first output time for providing the high-frequency ultrasonic wave and a second output time for providing the low-frequency ultrasonic wave.

2. The portable ultrasonic stimulator of claim 1, wherein the low-frequency ultrasonic wave is less than 1 MHz, and
wherein the high-frequency ultrasonic wave is greater than or equal to 1 MHz.

3. The portable ultrasonic stimulator of claim 1, wherein the high-frequency ultrasonic wave is provided to accelerate to produce a skin aging prevention material in skin and decelerate to produce a skin aging acceleration material in skin, and
wherein the low-frequency ultrasonic wave is provided to provide a temperature rise effect of a skin layer.

4. The portable ultrasonic stimulator of claim 1, wherein the controller provides the ultrasonic transducer with current intensity in which the high-frequency ultrasonic wave penetrates into the dermal layer.

5. The portable ultrasonic stimulator of claim 1, wherein the entire output time is set such that the sum of the second output time is longer than the sum of the first output time, and
wherein the ultrasonic transducer has maximum efficiency in the low-frequency ultrasonic wave.

6. The portable ultrasonic stimulator of claim 1, wherein the ultrasonic transducer is replaced and combined to the ultrasonic transducer combining part, such that a type of an ultrasonic wave output to skin is set.

7. The portable ultrasonic stimulator of claim 6, further comprising:
an ultrasonic generator,
wherein the ultrasonic transducer is selected among a point-focused ultrasonic transducer, a line-focused ultrasonic transducer, and a planar ultrasonic transducer.

8. The portable ultrasonic stimulator of claim 7, wherein the planar ultrasonic transducer is included in the ultrasonic generator,
wherein a surface of the ultrasonic generator is formed in a form being in direct contact with the skin of the user, and
wherein the planar ultrasonic transducer vibrates to output an ultrasonic wave of an unfocused form to the outside of the ultrasonic generator to be provided into the skin being in contact with the ultrasonic generator.

9. The portable ultrasonic stimulator of claim 7, wherein the point-focused ultrasonic transducer is formed in a shape, a cross section of which has a specific curvature, in the ultrasonic generator, and
wherein the ultrasonic generator allows an ultrasonic wave generated by the point-focused ultrasonic transducer to be focused on a center point according to the curvature of the point-focused ultrasonic transducer.

10. The portable ultrasonic stimulator of claim 7, wherein the line-focused ultrasonic transducer is formed in a shape, a cross section of which has a specific curvature, in the ultrasonic generator, and
wherein the ultrasonic generator allows an ultrasonic wave generated by the line-focused ultrasonic transducer to be focused on a specific point on a line, the specific point being a center point of the cross section.

11. The portable ultrasonic stimulator of claim 7, wherein the point-focused ultrasonic transducer or the line-focused ultrasonic transducer includes a cartridge housing, an acoustic liquid, and an acoustic window,
wherein the acoustic liquid functions as a medium allowing an ultrasonic wave generated by the point-focused ultrasonic transducer or the line-focused ultrasonic transducer to be delivered into a skin tissue of the user, and
wherein the acoustic window allows the acoustic liquid to be sealed in the cartridge housing and plays a role as a path where an ultrasonic wave provided by the point-focused ultrasonic transducer or the line-focused ultrasonic transducer to the skin of the user.

12. The portable ultrasonic stimulator of claim 1, wherein the controller sets the entire output time when it is able to reach a tightening effect, with regard to a temperature rise by a low-frequency ultrasonic wave during one or more second output times included in the entire output time and a temperature rise by a high-frequency ultrasonic wave during one or more first output times included in the entire output time.

13. The portable ultrasonic stimulator of claim 12, wherein the controller sets the entire output time based on a high-frequency ultrasonic wave output condition and a low-frequency ultrasonic wave output condition to calculate the shortest time when it is able to generate a skin tightening effect at a specific location, when the user uses a manner which repeats a process of placing the ultrasonic stimulator on each location of skin during a specific time to output an ultrasonic wave and moving the ultrasonic stimulator to a next location to output the ultrasonic wave.

14. The portable ultrasonic stimulator of claim 1, further comprising:
a motion sensor configured to measure motion of the portable ultrasonic stimulator,
wherein the controller sets the ratio of the first output time to the second output time with regard to a skin characteristic of the user and a purpose of use, when the user rubs a skin region to perform therapy or care while outputting an ultrasonic wave, sets a time length where it is able to increase the entire area of the skin region to a specific temperature or more to the entire output time, and sets the entire output time when it is able to reach a tightening effect, with regard to a temperature rise by the low-frequency ultrasonic wave during the second output time and a temperature rise by the high-frequency ultrasonic wave during the one or more first output times.

15. The portable ultrasonic stimulator of claim 14, wherein the controller determines whether the entire skin region of the user is evenly rubbed based on a sensing result of the motion sensor.
